(19) United States
(12) Patent Application Publication
(10) Pub. No.: US 2025/0228575 A1
(43) Pub. Date: Jul. 17, 2025

(54) DRILL GUIDE SYSTEM FOR DISTAL TENDON REPAIR

(71) Applicant: Skeletal Dynamics, Inc., Miami, FL (US)

(72) Inventors: Jorge L. Orbay, Miami, FL (US); Daniel A. Sixto, Miami, FL (US); Gabriel Mesa, Miami, FL (US); Nicolas E. Zedan, Pembroke Pines, FL (US); Roger Pisarnwongs, Santa Clarita, CA (US); Tom Weisel, Ventura, CA (US); Peter Evans, Palm Beach Gardens, FL (US)

(21) Appl. No.: 19/016,865

(22) Filed: Jan. 10, 2025

Related U.S. Application Data
(60) Provisional application No. 63/621,328, filed on Jan. 16, 2024.

(51) Int. Cl. A61B 17/17; A61B 17/04

(52) U.S. Cl. CPC A61B 17/1714; A61B 17/0401; A61B 17/0485; A61B 2017/0404

(57) ABSTRACT

A guide system and method for drilling through a bone and reattaching a tendon comprising a drill guide having a body section and a spike section, a locking rod, a suture passing guide, a suture passing needle, and suture loop; the body section comprising a locking rod ratchet gear and a locking rod tunnel adapted to receive the locking rod; the spike section comprising a hook arm terminating in a hole adapted to receive the suture passing needle and a spike adapted to engage a distal side of the bone; the locking rod comprising a sharp tip adapted to engage a proximal side of the bone, a suture passing guide tunnel adapted to receive the suture passing guide, and a plurality of outer ratchet teeth adapted to engage the locking rod ratchet gear; the suture passing guide comprising a suture passing needle tunnel adapted to receive the suture passing needle.

### CLAIM OF PRIORITY

This application is being filed as a non-provisional patent application under 35 U.S.C. § 111(a) and 37 CFR § 1.53(b). This application claims priority under 35 U.S.C. § 119(e) to U.S. provisional patent application Ser. No. 63/621,328 filed on Jan. 16, 2024, the contents of which are incorporated herein by reference.

### TECHNICAL FIELD

The technical field relates generally to devices and systems for soft tissue repair in orthopedic procedures and, in particular, to a drill guide system adapted to repair a distal biceps rupture to its anatomical insertion point through a single incision approach.

### BACKGROUND

The distal biceps tendon is a tissue that attaches the biceps muscle to the radial tuberosity of the radius bone and improves supination of the forearm. Overuse or sudden injury can result in a distal biceps tendon rupture if the tendon tears from its attachment site. When a rupture is left untreated, supination strength can be reduced.

In order to treat a distal biceps tendon rupture, surgical repair may be required to repair the distal biceps tendon to its anatomical insertion point. However, accessing the anatomical insertion point can be difficult. Current techniques involve either a double-incision or a single-incision approach in the surgical repair of a distal tendon rupture.

Most existing techniques use the double-incision method. However, a common complication from the double-incision technique is heterotopic ossification. While a single-incision approach can reduce the risk of heterotopic ossification, existing instruments have restricted operational angles making anatomic reconstruction challenging. Furthermore, existing single-incision techniques present a risk of peripheral nerve injury.

Therefore, there exists a need for a system that allows for a single incision approach that assists the surgeon in accessing the anatomical insertion point of the distal biceps tendon without damaging the radial nerve.

### SUMMARY

It is among the objects of the present system to overcome the limitations of the heretofore-known devices by providing inventive features to achieve: a.) a single-incision technique that provides access to the anatomical insertion point of the distal biceps tendon; b.) compact instrumentation; and c.) tendon repair and reattachment without damage to the radial nerve.

It is an object of the present system to provide a guide system adapted for drilling through a bone to facilitate a tendon repair and reattachment. In one embodiment, the guide system comprises a drill guide having a body section proximal to a spike section, a locking rod, a suture passing guide, a suture passing needle, and a suture loop.

The body section of the drill guide comprises a locking rod tunnel, a locking rod ratchet gear, and a guide arm. The locking rod tunnel further comprises a proximal end and a distal end. The spike section of the drill guide comprises a hook arm that terminates in a hole and an optional spike. The spike is adapted to engage a distal side of the bone.

The locking rod is adapted to be received by the locking rod tunnel of the drill guide. The locking rod comprises a proximal end, a distal end, and a shaft therebetween. The proximal end of the locking rod optionally further comprises a push pad. The distal end of the locking rod optionally further comprises a sharp tip adapted to engage a proximal side of the bone. The shaft comprises a suture passing guide tunnel bored through the shaft.

The shaft of the locking rod may further comprise a plurality of outer ratchet teeth. The locking rod ratchet gear of the drill guide is adapted to engage the plurality of outer ratchet teeth of the locking rod to permit the locking rod to slide freely in a direction from the proximal end to the distal end of the locking rod tunnel, and to prevent the locking rod from sliding in the direction from the distal end to the proximal end of the locking rod tunnel.

In at least one further embodiment, the body section of the drill guide may further comprise a locking rod button adapted to disengage the locking rod ratchet gear from the plurality of outer ratchet teeth on the shaft of the locking rod and permit the locking rod to slide in the direction from the distal end to the proximal end of the locking rod tunnel.

The suture passing guide is adapted to be received by the suture passing guide tunnel that is bored through the shaft of the locking rod. The suture passing guide comprises a proximal end, a distal end, and a shaft therebetween. The shaft of the suture passing guide further comprises a suture passing needle tunnel.

In some embodiments, the hole of the hook arm of the drill guide is optionally a stop-hole. In at least some further embodiments having a stop-hole, the shaft of the suture passing guide has a diameter larger than a diameter of the stop-hole of the drill guide hook arm and is adapted to be stopped by the stop-hole when the suture passing guide passes through the suture passing guide tunnel.

The suture passing needle is adapted to be received by the suture passing needle tunnel of the suture passing guide and to pass through the hole on the hook arm of the drill guide. The suture passing needle comprises a leading end, a trailing end, and a substantially cylindrical body therebetween. The leading end optionally further comprises a tip end. The trailing end is optionally adapted to receive a suture loop. The suture loop may have an open loop adapted to receive a suture.

In at least one further embodiment, the suture passing guide tunnel of the locking rod is adapted to receive a drill and to guide the drill in the direction of the hook arm hole. In embodiments wherein the hole of the hook arm is a stop-hole, the suture passing guide tunnel of the locking rod may be adapted to receive a drill having a diameter larger than a diameter of the stop-hole of the drill guide, and to guide the drill in the direction of the stop-hole, the stop-hole being adapted to limit the travel of the drill through the suture passing guide tunnel of the locking rod.

In at least one other embodiment, the shaft of the locking rod may further comprise a channel at the distal end of the locking rod, and the body section of the drill guide may further comprise a pin adapted to intersect with the locking rod tunnel. The pin is further adapted to engage the channel on the shaft of the locking rod to secure and prevent rotation of the locking rod when the locking rod is received by the locking rod tunnel.

In at least one further embodiment, the locking rod may further comprise a distal segment and a proximal segment. The distal segment is located at the distal end of the locking rod. The distal segment comprises a distal end, a proximal end, and a shaft therebetween with a distal segment tunnel bored through the shaft. In such an embodiment, the distal end of the distal segment comprises the sharp tip of the locking rod, and the shaft of the distal segment further comprises the channel adapted to be engaged by the pin of the body section of the drill guide. The proximal segment is located at the proximal end of the locking rod. The proximal segment comprises a distal end, a proximal end, and a shaft therebetween with a proximal segment tunnel bored through the shaft. In such embodiments, the proximal end of the proximal segment may comprise the optional push pad of the locking rod, and the shaft of the proximal segment further comprises the plurality of outer ratchet teeth of the locking rod. The proximal end of the distal segment and the distal end of the proximal segment are adapted to removably attach to form the locking rod. The distal segment tunnel and the proximal segment tunnel are adapted to align to form the suture passing guide tunnel when the distal segment and the proximal segment are attached.

In at least some embodiments wherein the locking rod comprises a distal segment and proximal segment, the distal end of the proximal segment comprises an internal thread adapted to rotationally engage an external thread of the proximal end of the distal segment. In further embodiments, the channel and the pin may be adapted to prevent rotation of the distal segment while allowing linear translation of the distal segment and rotation of the proximal segment.

It is a further object to provide a method of using a guide system to facilitate drilling through a bone and tendon repair and reattachment. In at least one embodiment, the method comprises placing a hook arm of a drill guide around the bone. The drill guide may comprise a hook arm terminating in a hole, the hole may be adapted to align with a distal side of the bone. The drill guide may further comprise a body section having a locking rod tunnel. Next, a locking rod may be inserted into the locking rod tunnel of the drill guide until the locking rod contacts a proximal side of the bone. The locking rod may comprise a suture passing guide tunnel.

Thereafter, a drill may be inserted into the suture passing guide tunnel of the locking rod and then may drill through the bone to form a bone tunnel from the proximal side to the distal side of the bone. The drill may then be removed from the bone and the suture passing guide tunnel.

Next, a suture passing guide may be inserted into the suture passing guide tunnel of the locking rod and through the bone tunnel. The suture passing guide may comprise a suture passing needle tunnel. A suture passing needle may then be inserted into the suture passing needle tunnel of the suture passing guide in the bone tunnel. The suture passing needle may be adapted to receive a suture.

Thereafter, the suture passing guide may be removed from the bone tunnel and the suture passing guide tunnel of the locking rod while leaving the suture passing needle and the suture in the bone tunnel. The locking rod may then be removed from the locking rod tunnel of the drill guide and the drill guide may be removed from the bone thereafter.

In at least some further embodiments, the method may further comprise engaging a sharp tip of the locking rod with the proximal side of the bone when inserting a locking rod into the locking rod tunnel of the drill guide until the locking rod contacts the proximal side of the bone, and disengaging the sharp tip of the locking rod prior to removing the locking rod from the locking rod tunnel of the drill guide. In some embodiments, the method may optionally further comprise engaging a spike on the hook arm with the distal side of the bone when placing the hook arm of the drill guide around the bone, and disengaging the spike on the hook arm prior to removing the drill guide from the bone.

In yet another embodiment, the step of inserting the locking rod into the locking rod tunnel may further comprise engaging one of a plurality of outer ratchet teeth on a shaft of the locking rod with a locking rod ratchet gear on the drill guide body section. In such embodiments, the method may further comprise disengaging the locking rod from the drill guide by pushing on a locking rod button on the drill guide body section to release the locking rod ratchet gear of the drill guide prior to removing the locking rod from the locking rod tunnel.

It is another object of the system to provide a button threader assembly suitable for threading a suture through a suture button. In at least one embodiment, the button threader assembly comprises a suture button, a button threader handle, and a button threader holder.

The suture button comprises two holes. The two holes are each adapted to receive a suture loop. The button threader handle comprises two substantially pliable loops. The two substantially pliable loops are each adapted to compress through each of the corresponding two holes of the suture button.

The button threader holder comprises a proximal end, a distal end, and a body therebetween. The proximal end of the button threader holder further comprises two arms and a button slot. The two arms of the proximal end are adapted to receive the button threader handle. The button slot is adapted to receive the suture button. The body of the button threader holder further comprises two channels adapted to receive the two loops of the button threader handle when each of the two loops is inserted into each of the corresponding two holes of the suture button.

In at least one further embodiment, each of the two substantially pliable loops of the button threader handle is adapted to receive a suture loop when the button threader handle is received by the two arms of the button threader holder and the two substantially pliable loops are inserted into each of the corresponding two holes of the suture button. The button threader handle is further adapted to simultaneously pull the two substantially pliable loops, each holding a suture loop, through each of the corresponding two holes of the suture button when the button threader handle is detached from the two arms of the button threader holder.

Although the system is illustrated and described herein as embodied for use in distal bicep tendon repair, it is nevertheless not intended to be limited to only the details or use shown, since various modifications and structural changes may be made therein without departing from the spirit of the invention and within the scope and range of equivalents of the claims.

The construction of the invention, however, together with additional objects and advantages thereof will be best understood from the following description of the specific disclosed embodiments when read in connection with the accompanying drawings.

### BRIEF DESCRIPTION OF THE DRAWINGS

FIGS. 1 and 2 illustrate, respectively, a side view and an enlarged view of a guide system adapted for drilling through a bone in accordance with one embodiment.

FIG. 3 illustrates an exploded view of the guide system of FIGS. 1 and 2 showing a drill guide, a locking rod, a suture passing guide, a suture passing needle, and a suture loop in accordance with one embodiment.

FIG. 4 illustrates an enlarged exploded view of the guide system of FIG. 3, showing additional details in accordance with one embodiment.

FIGS. 5 and 6 illustrate enlarged isometric views of the guide system of FIGS. 1 and 2 in accordance with one embodiment.

FIGS. 7 and 8 illustrate isometric views of the guide system engaged with a bone in accordance with one embodiment.

FIG. 9 illustrates a side view of a drill guide in accordance with one embodiment.

FIGS. 10 and 11 illustrate isometric views of the drill guide of FIG. 9 showing additional features in accordance with one embodiment.

FIG. 12 illustrates a cross-sectional side view of the drill guide of FIG. 9 showing additional features in accordance with one embodiment.

FIG. 13 illustrates a side view of a locking rod in accordance with one embodiment.

FIGS. 14 and 15 illustrate isometric views of the locking rod of FIG. 13 showing additional features in accordance with one embodiment.

FIG. 16 illustrates a cross-sectional view of the locking rod of FIG. 13 showing additional features in accordance with one embodiment.

FIG. 17 illustrates a side view of a drill guide having received a locking rod through a locking rod tunnel of the drill guide in accordance with one embodiment, wherein the drill guide and the locking rod are shown, for illustrative purposes only, as transparent to show additional features.

FIG. 18 illustrates a side view of a suture passing guide in accordance with one embodiment.

FIGS. 19 and 20 illustrate isometric views of the suture passing guide of FIG. 18 showing additional features in accordance with one embodiment.

FIG. 21 illustrates a cross-sectional view of the suture passing guide of FIG. 18 showing additional features in accordance with one embodiment.

FIG. 22 illustrates an isometric view of a suture passing needle having received a suture loop in accordance with one embodiment.

FIG. 23A illustrates an enlarged exploded view of the suture passing needle an end of the suture loop of FIG. 22 showing additional features in accordance with one embodiment.

FIG. 23B illustrates an enlarged view of a leading end of the suture passing needle of FIGS. 22 and 23A showing additional features in accordance with one embodiment.

FIG. 24 illustrates an enlarged view of the suture loop of FIG. 22 showing additional features in accordance with one embodiment.

FIG. 25 illustrates a side view of a drill received by a locking rod that is received by a drill guide in accordance with another embodiment.

FIG. 26 illustrates an exploded view of the drill, the locking rod, and the drill guide of FIG. 25 in accordance with one embodiment.

FIGS. 27 and 28 illustrate isometric views of the drill received by the locking rod that is received by the drill guide of FIG. 25 showing additional features in accordance with one embodiment.

FIGS. 29 and 30 illustrate isometric views of the drill received by the locking rod that is received by the drill guide of FIG. 25 engaged with a bone in accordance with one embodiment.

FIG. 31 illustrates an isometric view of a button threader assembly in accordance with another embodiment.

FIG. 32 illustrates an exploded view of the button threader assembly of FIG. 31 showing a suture button, a button threader handle, and a button threader holder in accordance with one embodiment.

FIG. 33 illustrates an isometric view of a suture button in accordance with one embodiment.

FIG. 34 illustrates an isometric view of a button threader handle in accordance with one embodiment.

FIG. 35 illustrates an isometric view of button threader holder in accordance with one embodiment.

FIG. 36 illustrates an isometric view of the button threader assembly with two suture loops having been received by two loops of the button threader handle in accordance with another embodiment.

FIG. 37 illustrates an isometric view of the button threader assembly having threaded the two suture loops through two holes of the suture button in accordance with another embodiment.

FIG. 38 illustrates a side view of a drill guide with a pin having received a locking rod through the locking rod tunnel of the drill guide in accordance with one embodiment, wherein the drill guide and the locking rod are shown, for illustrative purposes only, as transparent to show additional features.

FIG. 39 illustrates a cross-sectional side view of the drill guide with the pin having received the locking rod of FIG. 38 showing additional features in accordance with one embodiment.

FIG. 40 illustrates an isometric view of a locking rod with a distal segment and a proximal segment attached in accordance with another embodiment.

FIG. 41 illustrates an exploded isometric view of the locking rod of FIG. 40 showing additional features in accordance with one embodiment.

FIG. 42 illustrates a cross-sectional exploded view of the locking rod of FIG. 41 showing additional features in accordance with one embodiment.

### DETAILED DESCRIPTION

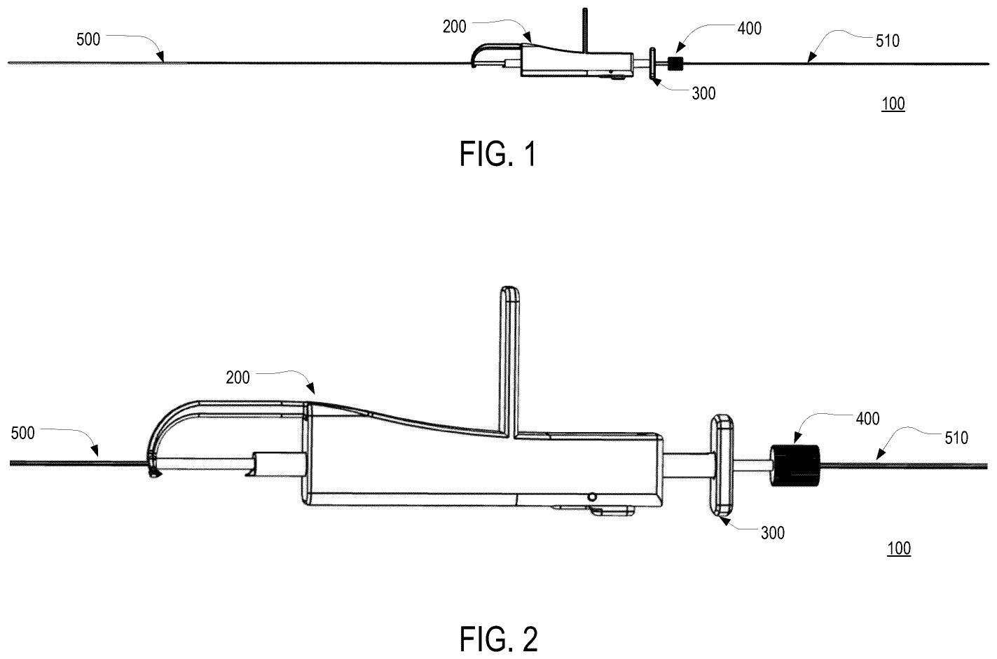

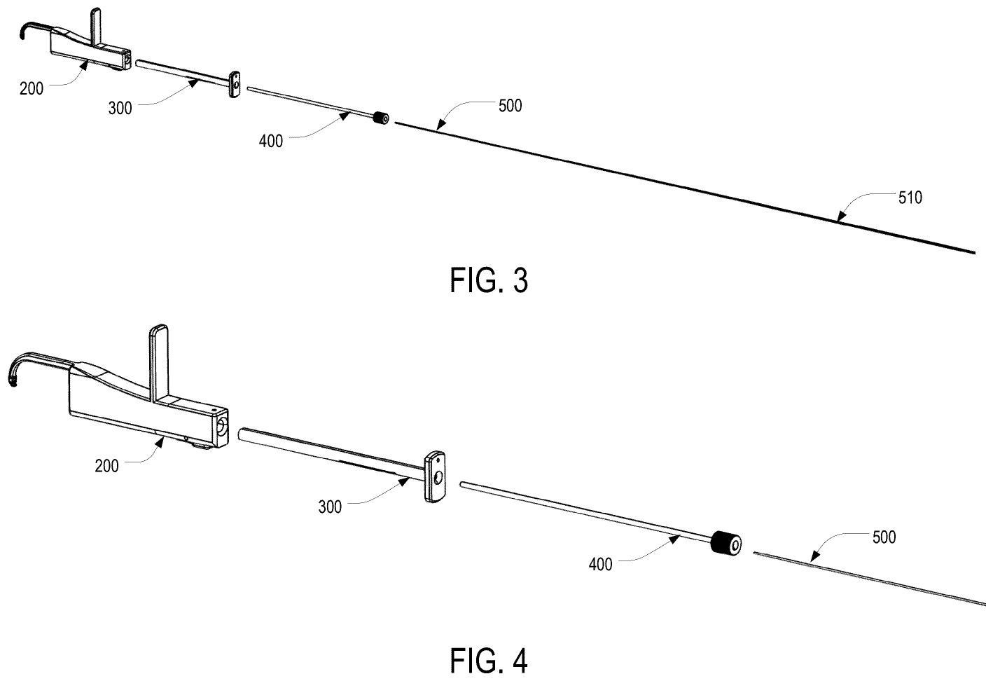

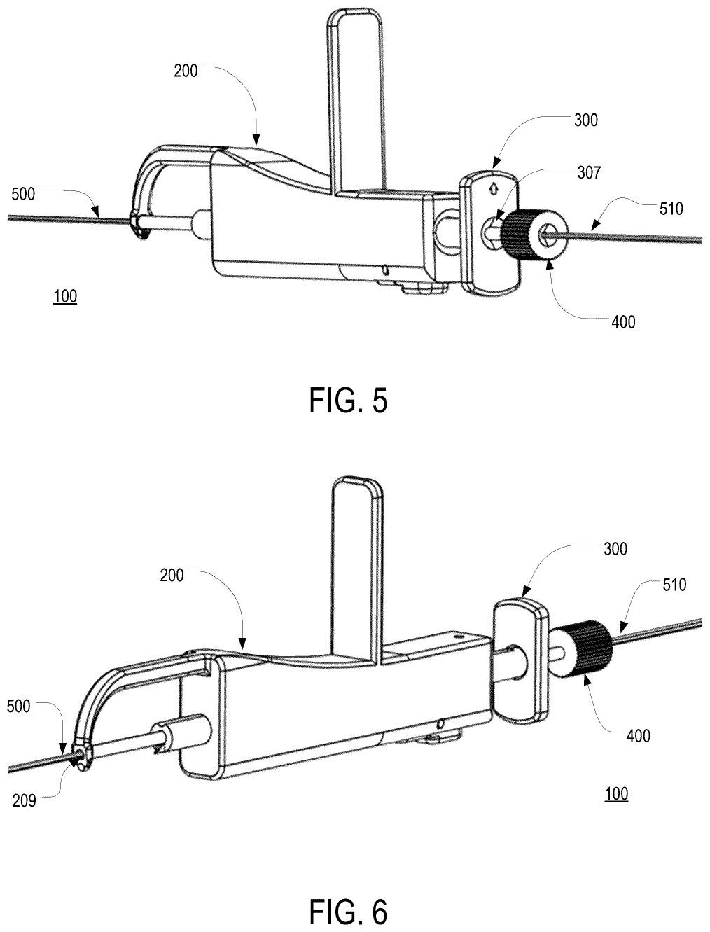

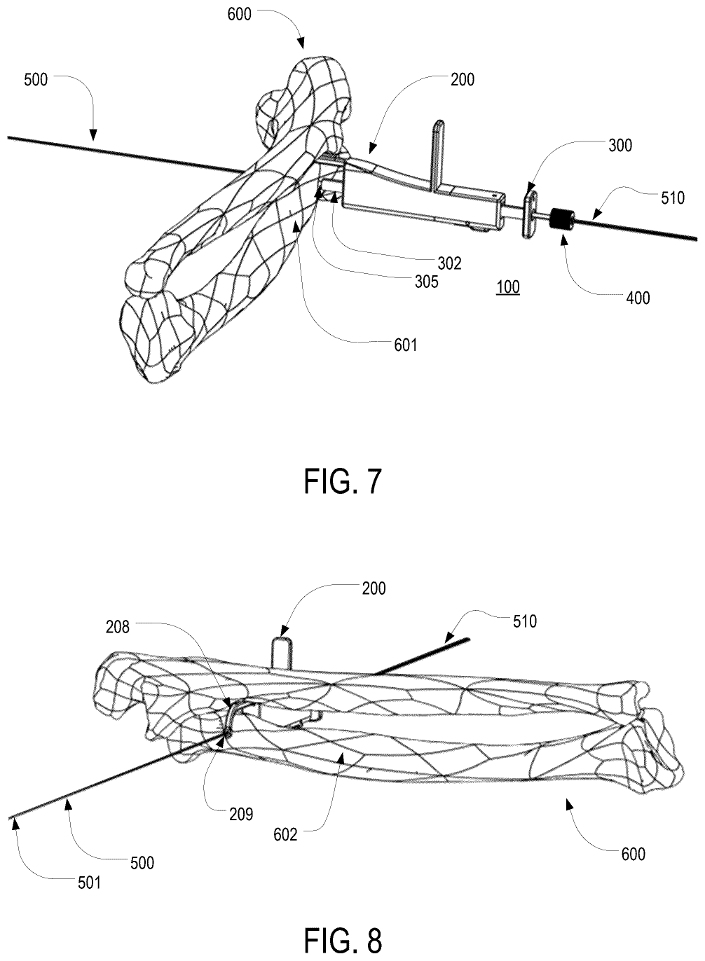

Referring to FIGS. 1 and 2, shown are a side view and an enlarged view, respectively, of a guide system (100) adapted for drilling through a bone (600) to facilitate tendon repair and reattachment. In accordance with at least one embodiment, the guide system (100) comprises a drill guide (200), a locking rod (300), a suture passing guide (400), a suture passing needle (500) and a suture loop (510). Referring to FIGS. 3 and 4, shown are an exploded view and an enlarged exploded view of the guide system (100) of FIGS. 1 and 2. Now referring to FIGS. 5 and 6, shown are various enlarged isometric views of the guide system (100) of FIGS. 1 and 2. Referring next to FIGS. 7 and 8, the guide system (100) is shown engaged with a bone (600) in accordance with one embodiment. For illustrative purposes only, the guide system (100) is shown engaged with the radial tuberosity of a radius. However, the present guide system (100) is not limited to this use or depiction.

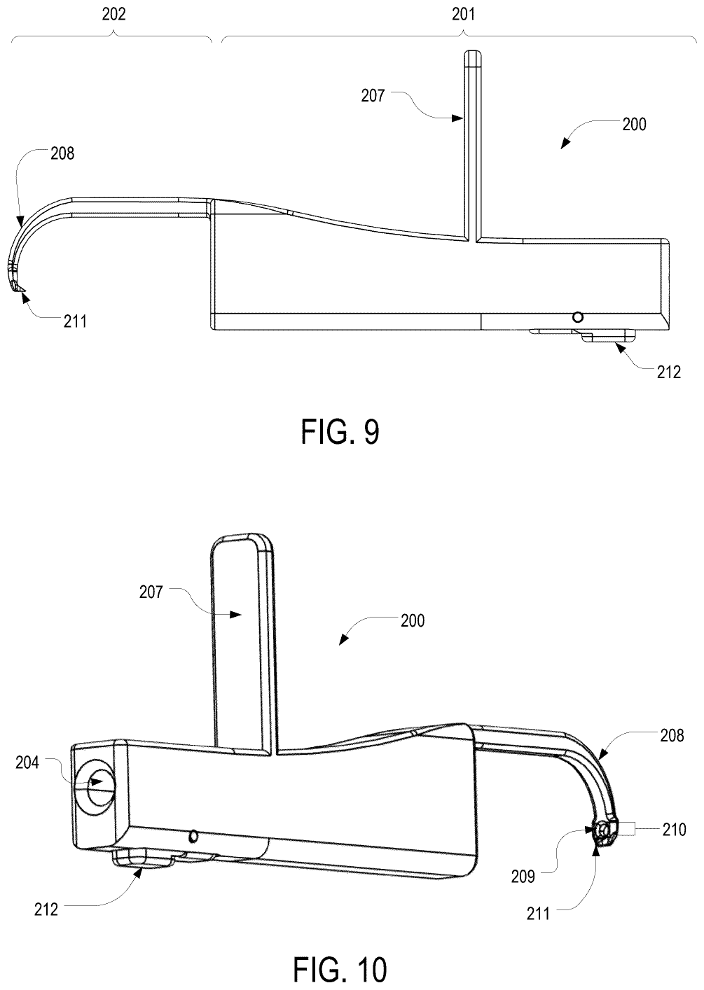

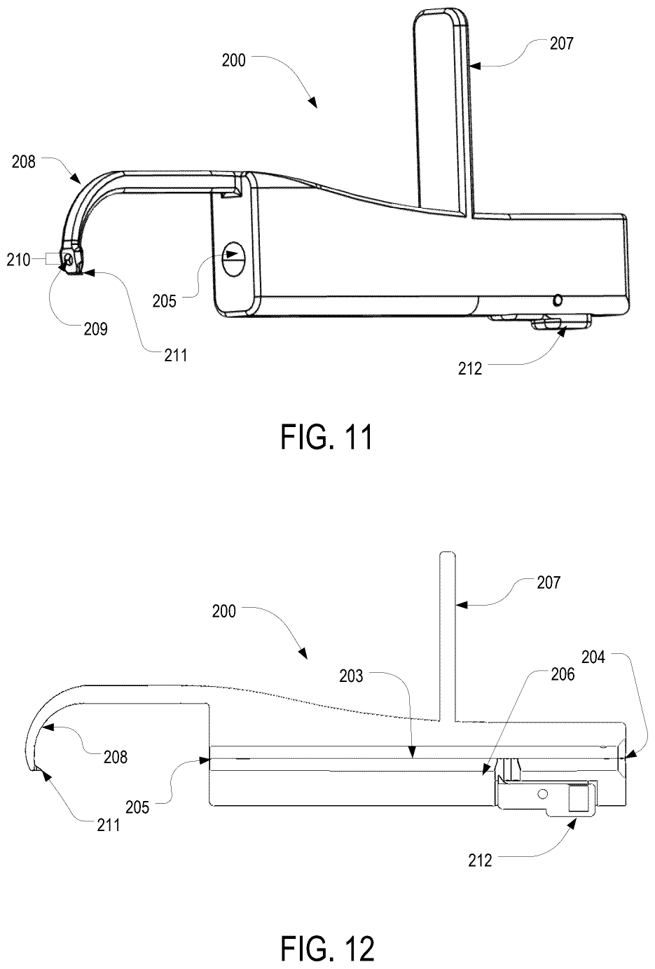

Referring now to FIGS. 9 through 12, shown are various views of a drill guide (200) in accordance with at least one embodiment. Referring to FIG. 9, the drill guide (200) comprises a body section (201) and a spike section (202), wherein the body section (201) is located proximal of the spike section (202). Referring again to FIGS. 9 and 12, the body section (201) of the drill guide (200) further comprises a locking rod tunnel (203), a locking rod ratchet gear (206), and a guide arm (207). Referring now to FIGS. 10, 11, and 12, the locking rod tunnel (203) further comprises a proximal end (204) and a distal end (205).

Referring again to FIGS. 9, 10, and 11, the spike section (202) of the drill guide (200) further comprises a hook arm (208). The hook arm (208) optionally terminates in a hole (209) having a diameter (210) and an optional spike (211). In at least some embodiments, the hole (209) of the hook arm (208) may optionally be a stop-hole. The spike (211) of the hook arm (208) is adapted to engage a distal side (602) of the bone (600), as further shown in FIG. 8. Although depicted and described herein as a spike (211), a spike (211) in accordance with at least one embodiment may comprise any sharp tip capable of biting into the bone (600). Such a sharp tip may include, but is not limited to, a prong, a point, a plurality of sharp points, etc.

In at least one further embodiment, the hook arm (208) may comprise a curvature adapted to match the curvature of the bone (600). In the case of a distal tendon repair and reattachment, the curvature of the hook arm (208) may be adapted to match the curvature of the radial tuberosity. In a further embodiment, the curvature of the hook arm (208) may be adapted to fit on tuberosity diameter sizes between 11-25 mm, or a full range of patient anatomies.

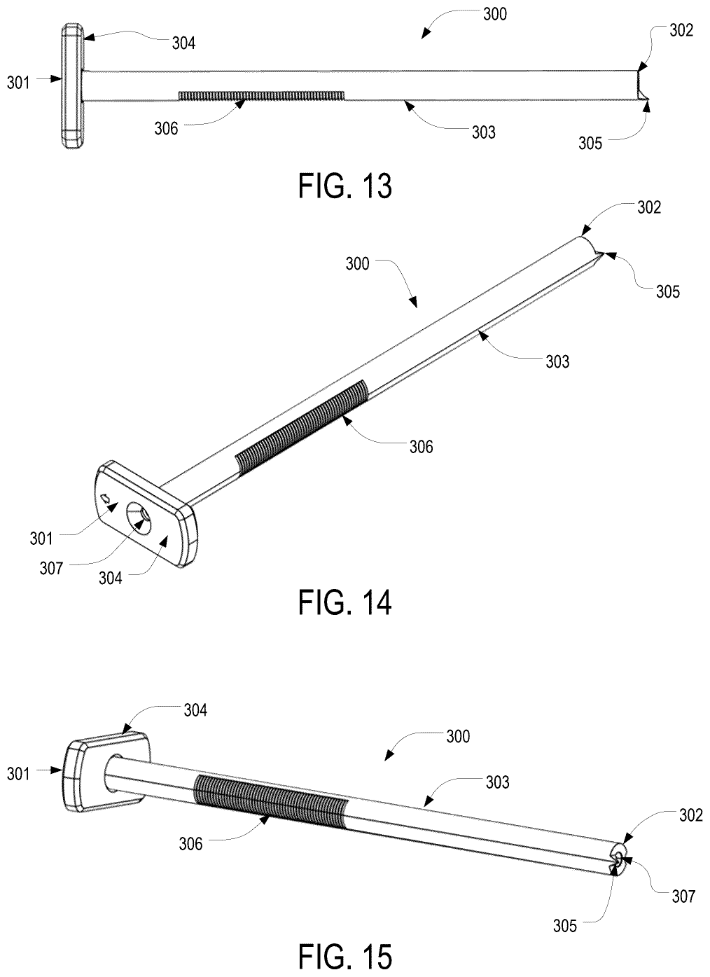

Referring now to FIGS. 13 through 16, shown are various views of a locking rod (300) in accordance with at least one embodiment. The locking rod (300) comprises a proximal end (301), a distal end (302), and a shaft (303) therebetween. In some embodiments, such as the ones depicted in the figures, the shaft (303) of the locking rod (300) may optionally be substantially cylindrical. In other embodiments, the shaft (303) may be other shapes. The proximal end (301) of the locking rod (300) may optionally further comprise a push pad (304). The distal end (302) of the locking rod (300) optionally further comprises a sharp tip (305) adapted to engage a proximal side (601) of the bone (600), as further shown in FIG. 7. Referring again to FIG. 7, the sharp tip (305) of the locking rod (300) may be adapted to lock the position of the drill guide (200) on the bone (600) when the sharp tip (305) engages the proximal side (601) of the bone (600). Although the sharp tip (305) is depicted as a single sharp tip (305), a sharp tip (305) in accordance with at least one embodiment may comprise a prong or a plurality of sharp tips.

The locking rod (300) is adapted to be received by the locking rod tunnel (203) of the body section (201) of the drill guide (200), as shown in the assembled guide system (100) in FIGS. 1, 2, and 5 through 7. Referring now to FIG. 17, shown is a side view of the drill guide (200) having received the locking rod (300) through the locking rod tunnel (203) of the drill guide (200) body section (201). The drill guide (200) and the locking rod (300) in FIG. 17 are depicted as transparent, for illustrative purposes only, to show the interaction between the locking rod (300) and the drill guide (200). The push pad (304) of the locking rod (300) proximal end (301) is adapted to permit pressure to be applied to the proximal end (301) of the locking rod when inserting the locking rod (300) into the locking rod tunnel (203) of the drill guide (200) body section (201). The shaft (303) of the locking rod (300) is adapted to be received by the proximal end (204) through to the distal end (205) of the locking rod tunnel (203).

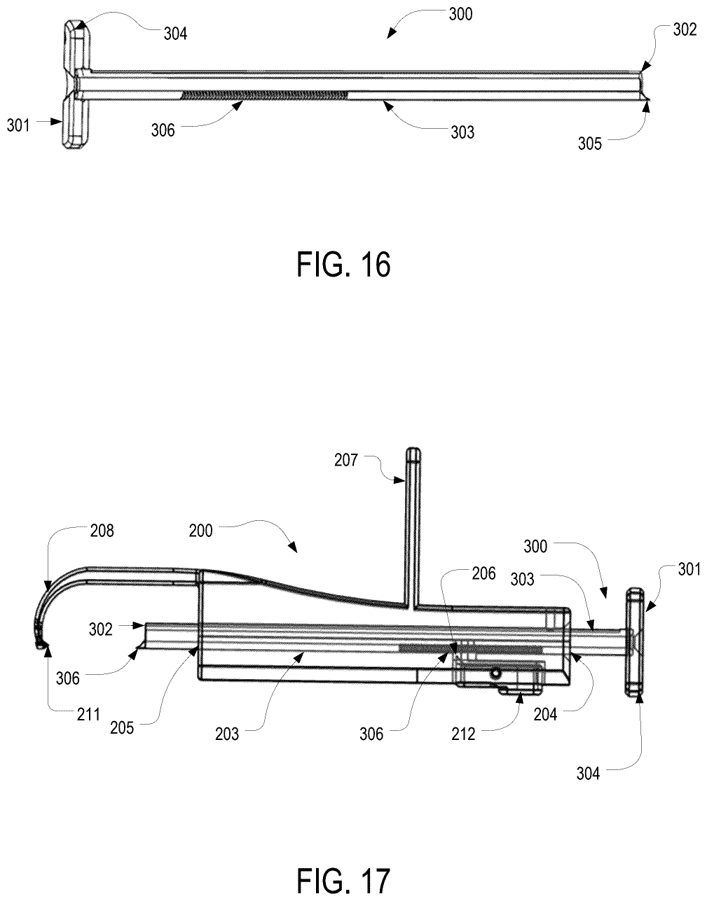

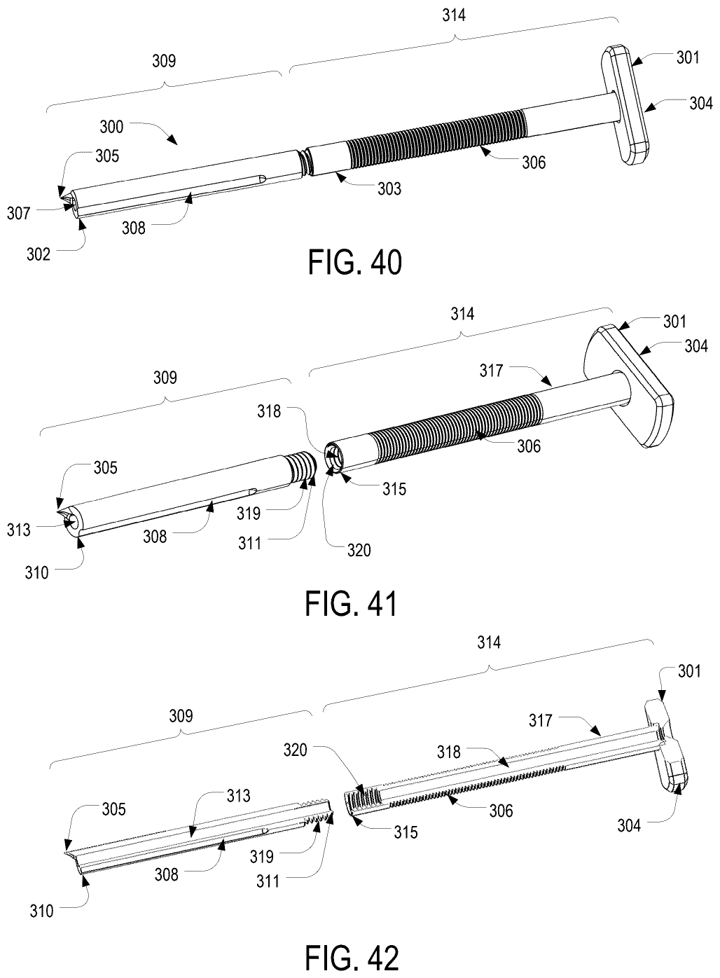

Referring again to FIGS. 13 through 16, the shaft (303) of the locking rod (300) optionally further comprises a plurality of outer ratchet teeth (306) and a suture passing guide tunnel (307) bored through the shaft (303), as shown in the cross-sectional view of FIG. 16. The plurality of outer ratchet teeth (306) may run along a complete circumference (see FIGS. 40 through 42) or a partial circumference (see FIGS. 13 through 16) of the shaft (303). Referring again to FIG. 17, the locking rod ratchet gear (206) of the drill guide (200) body section (201) is adapted to engage the plurality of outer ratchet teeth (306) on the shaft (303) of the locking rod (300). The locking rod ratchet gear (206) is adapted to permit the locking rod (300) to slide freely in a direction from the proximal end (204) to the distal end (205) of the locking rod tunnel (203). The locking rod ratchet gear (206) is further adapted to prevent the locking rod (300) from sliding in the direction from the distal end (205) to the proximal end (204) of the locking rod tunnel (203).

In at least one other embodiment, the body section (201) of the drill guide (200) further comprises a locking rod button (212), as shown in FIGS. 9 through 12. Now referring again to FIG. 17, the locking rod button (212) is adapted to disengage the locking rod ratchet gear (206) of the drill guide (200) body section (201) from the plurality of outer ratchet teeth (306) on the shaft (303) of the locking rod (300), and permit the locking rod (300) to slide freely in the direction from the distal end (205) to the proximal end (204) of the locking rod tunnel (203). Although the locking rod button (212) is depicted and described herein as a button, a locking rod button (212) in accordance with the present system may comprise a switch, a latch, or any means suitable for permitting the disengagement of the locking rod ratchet gear (206).

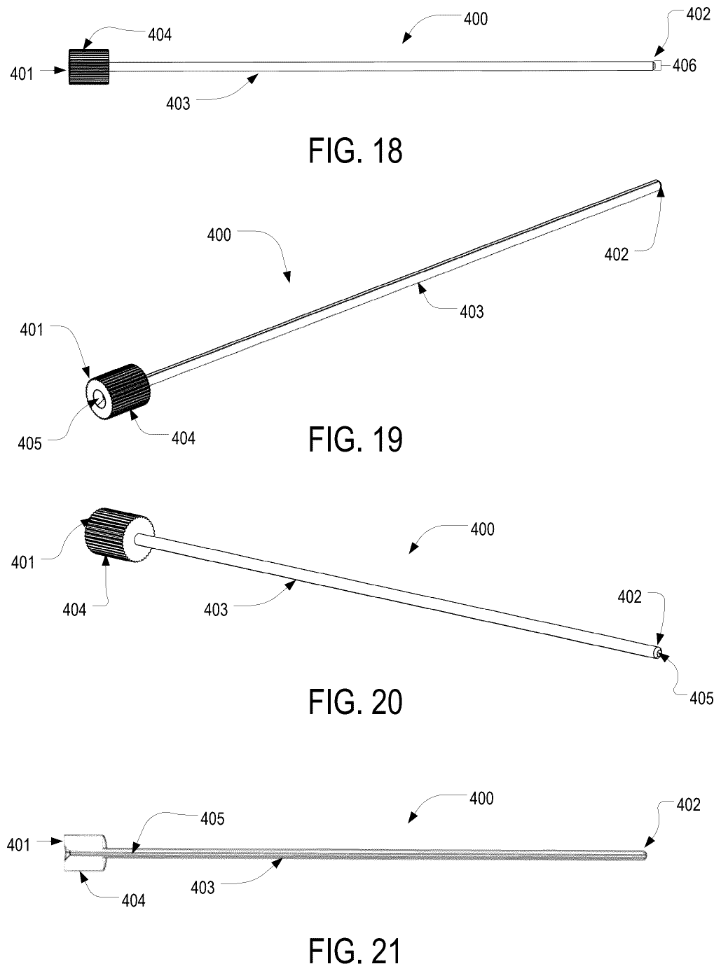

Referring next to FIGS. 18 through 21, shown are various views of a suture passing guide (400) in accordance with at least one embodiment. The suture passing guide (400) is adapted to be received by the suture passing guide tunnel (307) that is bored through the shaft (303) of the locking rod (300), as shown in the assembled guide system (100) of FIGS. 1, 2, and 5 through 7. Referring again to FIGS. 18 through 21, the suture passing guide comprises a proximal end (401), a distal end (402), and a shaft (403) therebetween. In some embodiments, such as the ones depicted in the figures, the shaft (403) is substantially cylindrical. In other embodiments, the shaft (403) may be other shapes. In at least some embodiments, the proximal end (401) of the suture passing guide (400) optionally further comprises a knob (404). The shaft (403) of the suture passing guide (400) further comprises a suture passing needle tunnel (405), as shown in the cross-sectional view of the suture passing guide (400) in FIG. 21.

Referring again to FIG. 18, in embodiments wherein the hole (209) of the hook arm (208) is a stop-hole, the shaft (403) of the suture passing guide (400) may optionally have a diameter (406) larger than a diameter (210) of the stop-hole (209) such that the suture passing guide (400) is adapted to be stopped by the stop-hole (209) of the drill guide (200) when the suture passing guide (400) passes through the suture passing guide tunnel (307) (See the assembled guide system (100) in FIG. 6).

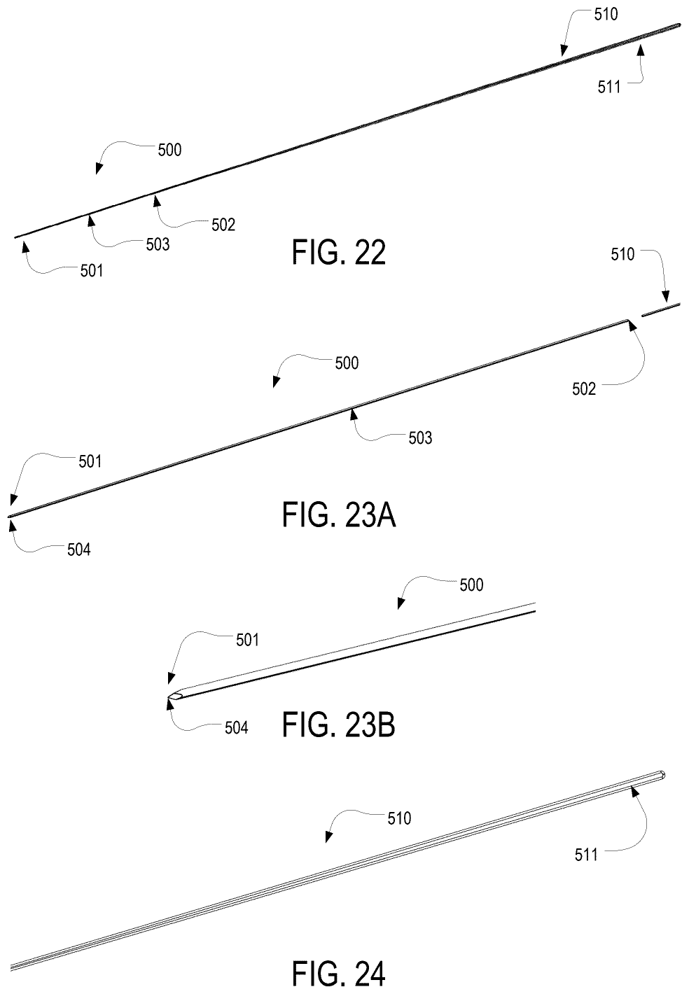

Referring now to FIG. 22, a suture passing needle (500) and suture loop (510) in accordance with at least one embodiment are shown. The suture passing needle (500) comprises a leading end (501), a trailing end (502), and a substantially cylindrical body (503) therebetween. Referring next to FIGS. 23A and 23B, the leading end (501) of the suture passing needle (500) optionally further comprises a tip end (504). The trailing end (502) of the suture passing needle (500) may be adapted to receive the suture loop (510). Now referring to FIGS. 22, 23A and 24, the suture loop (510) may have a distal end comprising a straight section and proximal end comprising an open loop (511) adapted to receive a suture, the suture may be attached to soft tissue. The straight section of the suture loop (510) is adapted to be received by and attached to the trailing end of the suture passing needle. The suture passing needle (500) and suture loop (510) are adapted to be received by the suture passing needle tunnel (405) of the suture passing guide (400) and to pass through the hole (209) on the hook arm (208) of the drill guide (200) (See the assembled guide system (100) in FIG. 6). Together, the suture passing needle (500) with the suture loop (510) and the guide system (100) aid in transferring the tendon to an anatomical insertion site during tendon repair and reattachment.

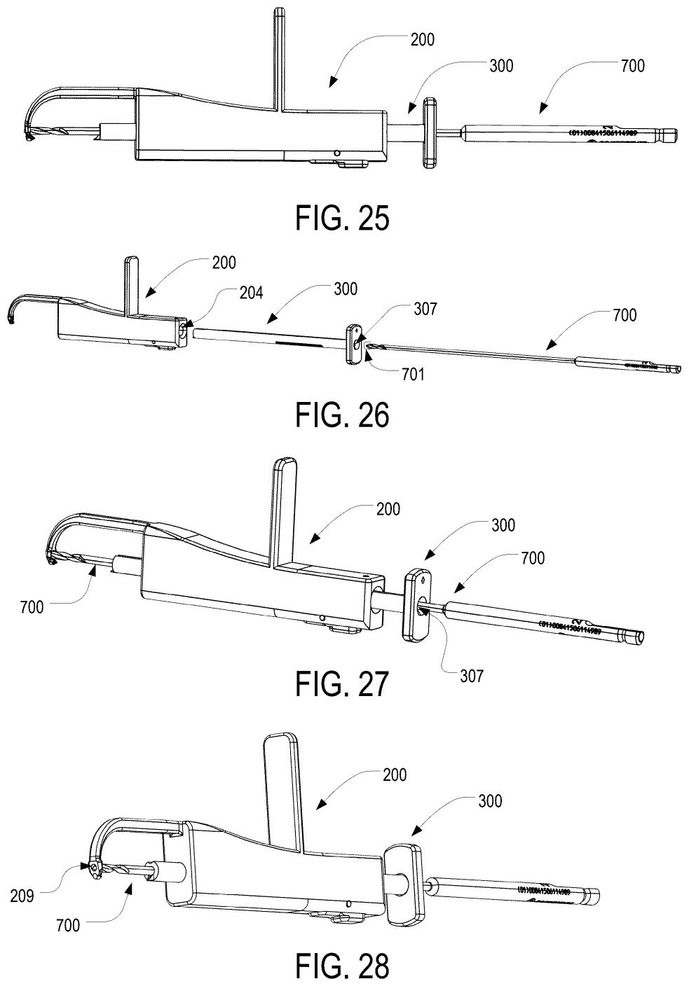

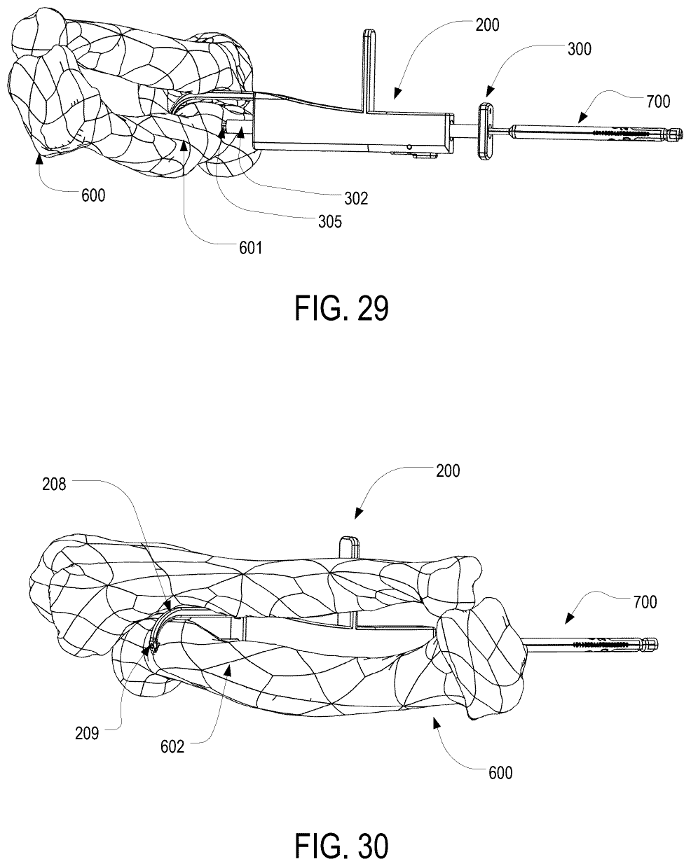

Now referring to FIGS. 25, 27, and 28, in a further embodiment, the suture passing guide tunnel (307) of the locking rod (300) is adapted to receive a drill (700) and to guide the drill (700) in the direction of the hook arm (208) hole (209) of the drill guide (200). In embodiments wherein the hook arm (208) hole (209) is a stop-hole, the drill (700) may optionally comprise a diameter (701) larger than a diameter (210) of the stop-hole (209) on the drill guide (200) such that the stop-hole (209) is adapted to limit the travel of the drill (700) through the suture passing guide tunnel (307) and prevent the drill (700) from passing through the stop-hole (209), as shown in FIGS. 27 and 28. FIG. 26 shows an exploded view of the drill guide (200), the locking rod (300), and the drill (700). Referring now to FIGS. 29 and 30, shown are various views of the drill guide (200) and the locking rod (300) having engaged with the bone (600), wherein the suture passing guide tunnel (307) of the locking rod (300) has received the drill (700).

In a further embodiment, the drill (700) is adapted to pass through the proximal side (601) to the distal side (602) of the bone (600), thereby creating a bone tunnel. In yet a further embodiment, the diameter (406) or width of the shaft (403) of the suture passing guide (400) is smaller than the diameter (701) of the drill (700) such that the suture passing guide (400) shaft (403) is adapted to pass through a bone tunnel formed by the drill (700).

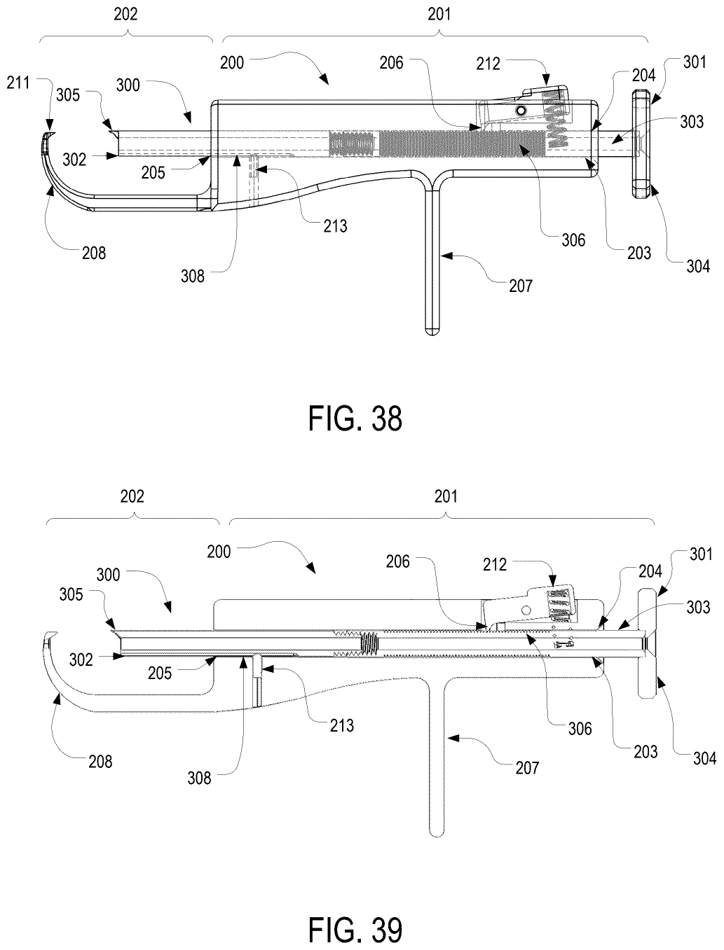

Referring now to FIGS. 38 and 39, in at least one embodiment, the shaft (303) of the locking rod (300) may optionally further comprise a channel (308) at the distal end (302) of the locking rod (300) (see also FIGS. 40 and 41), and the body section (201) of the drill guide (200) may optionally further comprise a removeable pin (213) that may be perpendicular to the axis of the locking rod tunnel (203). The drill guide (200) and the locking rod (300) in FIG. 38 are depicted as transparent, for illustrative purposes only, to show the interaction between the locking rod (300) and the drill guide (200). The pin (213) may be adapted to intersect with the locking rod tunnel (203). The pin (213) may be further adapted to engage the channel (308) on the shaft (303) of the locking rod (300) to secure and prevent rotation of the locking rod (300) when the locking rod (300) is received by the locking rod tunnel (203).

Now referring to FIGS. 40 through 42, in at least one further embodiment, the locking rod (300) may be separated into more than one segment. In one embodiment, the locking rod (300) may further comprise a distal segment (309) and a proximal segment (314). The distal segment (309) may be located at the distal end (302) of the locking rod (300). The distal segment (309) comprises a distal end (310), a proximal end (311), and a shaft (312) therebetween with a distal segment tunnel (313) bored through the shaft (312). The shaft (312) may optionally be substantially cylindrical. In such an embodiment, the distal end (310) of the distal segment (309) comprises the sharp tip (305) of the locking rod (300), and the shaft (312) of the distal segment (309) may optionally further comprise the channel (308) adapted to be engaged by the pin (213) of the body section (201) of the drill guide (200) (see FIG. 39). The proximal segment (314) is located at the proximal end (301) of the locking rod (300). The proximal segment (314) comprises a distal end (315), a proximal end (316), and a shaft (317) therebetween with a proximal segment tunnel (318) bored through the shaft (317). The shaft (317) may optionally be substantially cylindrical. In such an embodiment, the proximal end (316) of the proximal segment (314) comprises the optional push pad (304) of the locking rod (300), and the shaft (317) of the proximal segment (314) further comprises the optional plurality of outer ratchet teeth (306) of the locking rod (300). In at least some embodiments, the channel (308) and the pin (213) are adapted to prevent rotation of the distal segment (309) while allowing linear translation of the distal segment (309) and rotation of the proximal segment (314). The push pad (304) of the locking rod (300) is adapted to linearly translate the distal segment (309) upon compression of the push pad (304), while the channel (308) and pin (213) prevent rotation of the distal segment (309). The push pad (304) is further adapted to rotate the proximal segment (314) upon rotation of the push pad (304). This arrangement creates large compressive forces allowing for engagement of the distal segment (309) of the locking rod (300) against the surface of the proximal side (601) of the bone (600). It is to be understood that the linear translation of the distal segment (309) and rotation of the proximal segment (314) may also be achieved in embodiments not having a push pad (304).

The proximal end (311) of the distal segment (309) and the distal end (315) of the proximal segment (314) are adapted to removably attach to form the locking rod (300) (see FIG. 40). In some embodiments, such as the one depicted in FIGS. 41 and 42, the proximal end (311) of the distal segment (309) may optionally have an external helical thread (319) and the distal end (315) of the proximal segment (314) may optionally have an internal helical thread (320) adapted to engage the external helical thread (319) of the proximal end (311) of the distal segment (309) to secure the distal segment (309) and the proximal segment (314) together and form the locking rod (300) (see FIG. 40). In at least some embodiments, the internal thread (320) of the distal end (315) of the proximal segment (314) is adapted to rotationally engage the external thread (319) of the proximal end (311) of the distal segment (309). In at least one other embodiment (not shown), the distal end (315) of the proximal segment (314) may optionally have an external helical thread and the proximal end (311) of the distal segment (309) may optionally have an internal helical thread adapted to engage the external helical thread of the distal end (315) of the proximal segment (314) to secure the distal segment (309) and the proximal segment (314) together and form the locking rod (300). In embodiments in which the locking rod (300) comprises a push pad (304), the push pad (304) may be adapted to aid in rotation of the proximal segment (314) so that the internal (320) or external (not shown) thread of the proximal segment (314) may rotationally engage the external (319) or internal (not shown) thread, respectively, of the distal segment (309). In other embodiments, the proximal end (311) of the distal segment (309) and the distal end (315) of the proximal segment (314) may comprise other attachment mechanisms.

Referring again to FIGS. 40 through 42, when attached, the shaft (312) of the distal segment (309) and the shaft (317) of the proximal segment (314) (see FIGS. 41 and 42) are adapted to form the shaft (303) of the locking rod (300) (shown in FIG. 40). The distal segment tunnel (313) and the proximal segment tunnel (318) (see FIGS. 41 and 42) are adapted to align to form the suture passing guide tunnel (307) when the distal segment (309) and the proximal segment (314) are attached (see FIG. 40).

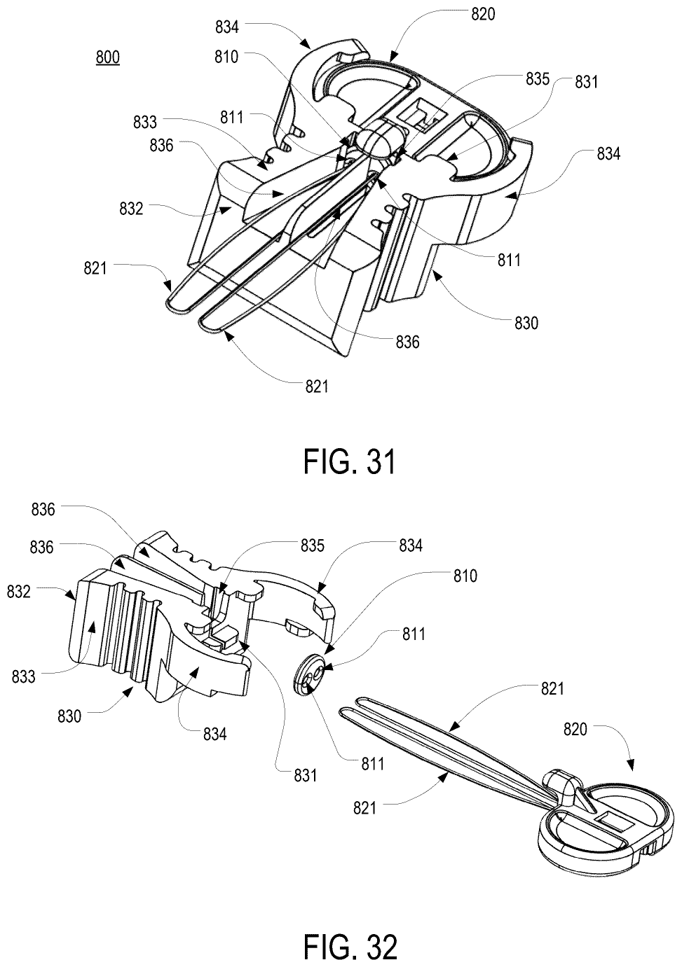

Now referring to FIG. 31, shown is a button threader assembly (800) suitable for threading a suture through a suture button (810), in accordance with another embodiment. The button threader assembly (800) comprises a suture button (810), a button threader handle (820), and a button threader holder (830). Referring to FIG. 32, an exploded view of the button threader assembly (800) is shown.

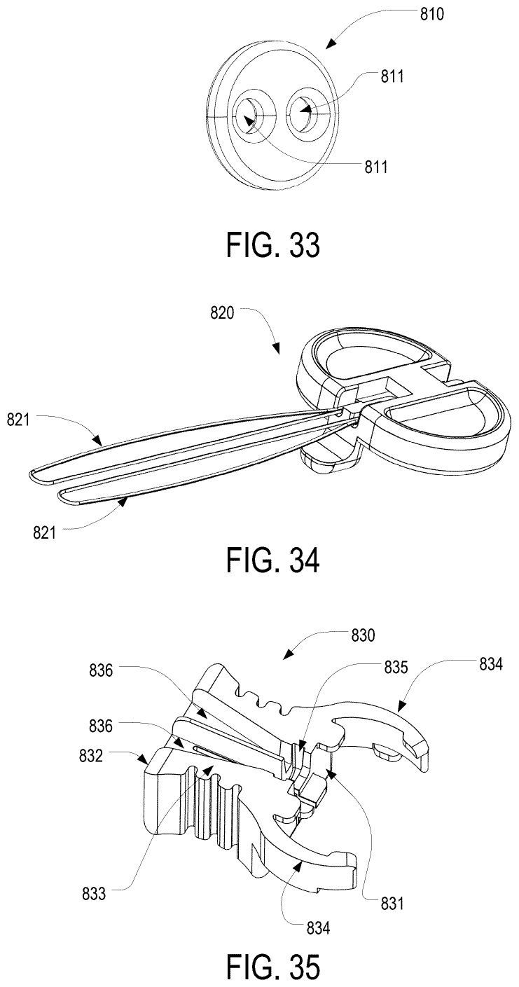

Referring now to FIG. 33, a suture button (810) in accordance with at least one embodiment of the present system is shown. The suture button (810) comprises two holes (811) each adapted to receive a suture loop (840), as shown in FIG. 37.

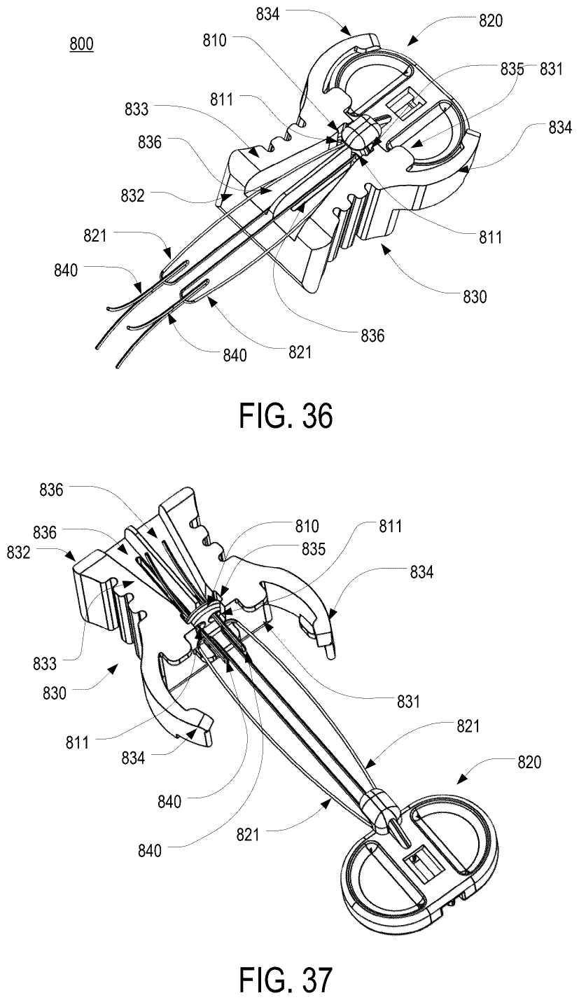

Referring next to FIG. 34, a button threader handle (820) in accordance with at least one embodiment of the present system is shown. The button threader handle (820) comprises two substantially pliable loops (821) each adapted to compress through each of the corresponding two holes (811) of the suture button (810), as shown in FIGS. 31 and 36.

Referring now to FIG. 35, a button threader holder (830) in accordance with at least one embodiment of the present system is shown. The button threader holder (830) comprises a proximal end (831), a distal end (832), and a body (833) therebetween. The proximal end (831) of the button threader holder (830) comprises two arms (834) adapted to receive the button threader handle (820), as depicted in FIGS. 31 and 36. Referring again to FIG. 35, the proximal end (831) of the button threader holder (830) further comprises a button slot (835) adapted to receive the suture button (810), as shown in FIGS. 31, 36, and 37. Again referring to FIG. 35, the body (833) of the button threader holder (830) further comprises two channels (836) adapted to receive the two loops (821) of the button threader handle (820) when each of the two loops (821) is inserted into each of the corresponding two holes (811) of the suture button (810), as shown in FIGS. 31 and 36.

Referring again to FIG. 36, in a further embodiment, each of the two substantially pliable loops (821) of the button threader handle (820) is adapted to receive the suture loop (840) when the button threader handle (820) is received by the two arms (834) of the button threader holder (830) and the two substantially pliable loops (821) of the button threader handle (820) are inserted into each of the corresponding two holes (811) of the suture button (810). Referring now to FIG. 37, in another embodiment, the button threader handle (820) is adapted to simultaneously pull each of the two substantially pliable loops (821) each holding a suture loop (840) through each of the corresponding two holes (811) of the suture button (810) when the button threader handle (820) is disengaged from the two arms (834) of the button threader holder (830).

It should be understood that in at least some embodiments of the button threader assembly (800), the suture button (810) may comprise one or more holes (811) each adapted to receive a suture loop (840), the button threader handle (820) may comprise one or more substantially pliable loops (821) each adapted to compress through a corresponding one of the one or more holes (811) of the suture button (810), and the button threader holder (830) may comprise one or more channels (836) each adapted to receive a corresponding one of the one or more substantially pliable loops (821) of the button threader handle (820).

In operation, the following is a listing of the steps typically taken to utilize the disclosed guide system (100) to drill through a bone (600) for distal biceps tendon repair and reattachment:
1. Position the patient with their arm on a hand table in full extension and supination. Make a 4-5 cm incision approximately 2 cm distal to the elbow crease and expose the subcutaneous tissues.
2. Use caution to identify and release the lateral antebrachial cutaneous nerve and retract laterally. Cauterize veins as needed. Avoid injury to the cephalic vein. Identify and retract the brachioradialis laterally. Identify and protect the radial nerve.
3. Identify the ruptured Distal Biceps tendon. If the tendon has retracted proximally and is not visible from the incision, palpate the bicep distally to locate the position of the tendon. Use a blunt retractor to allow for exposure above the elbow crease. Once located, grasp the tendon and pull it distally, freeing it from surrounding adhesions.
4. If a single suture repair technique is preferred, skip to Step 5. Identify the short and long head of the distal biceps tendon. The short head runs more medial in the arm and inserts distally on the bicipital tuberosity. The long head runs more lateral in the arm and inserts proximally on the bicipital tuberosity. Use a #2 Suture Loop, ST Needle, White-Blue and #2 Suture Loop, ST Needle, White-Black to whipstitch the individual tendon heads spanning approximately 2 cm in length. Make sure to note which color suture represents the short and the long head.
5. Use a #2 Suture Loop, ST Needle, to prepare the tendon using a whipstitch technique spanning approximately 2 cm in length.
6. Use the Volar Capsule Elevator to elevate the distal tendon fibers, periosteum, and soft tissue dorsal to the tuberosity.
7. Position and hold the forearm in supination. Slide the drill guide (200) between the radius and ulna at the level of the bicipital tuberosity (FIGS. 29 and 30). Rotate the forearm to approximately 45 degrees of supination to allow space for the hook arm (208) to fit between the radius and ulna. Locate the drill guide (200) spike (211) just dorsal to the biceps insertion. Pronate the forearm from the supinated position until the radial tuberosity sits in the hook arm (208) and engages the spike (211). Use the locking rod (300) to lock the drill guide (200) in place at an approximately neutral position (FIGS. 29 and 30) by inserting the locking rod (300) into the locking rod tunnel (203) of the drill guide (200) until the sharp tip (305) of the locking rod (300) engages the proximal side (601) of the bone (600) (FIG. 29).
8. Insert a 2.0 mm drill (700) into the suture passing guide tunnel (307) of the locking rod (300) that is inserted in the locking rod tunnel (203) of the drill guide (200) and create a bi-cortical bone tunnel (FIG. 29). Make sure the drill (700) clears the far cortex. The optional stop-hole (209) on the hook arm (208) of the drill guide (200) prevents over drilling (FIGS. 28 through 30). Remove the drill (700). Leave the drill guide (200) and the locking rod (300) in place in preparation for the suture passing guide (400) and suture passing needle (500). If the short and long head repair is being completed, repeat this step twice. Ensure the bone tunnels created are approximately 12 mm apart to account for the two buttons that will be placed on the tuberosity. Center the guide system (100) on the tuberosity for a single-button repair.
9. Insert the suture passing guide (400) into the suture passing guide tunnel (307) of the locking rod (300) that is inserted in the locking rod tunnel (203) of the drill guide (200) until it has passed through both cortices (FIGS. 7 and 8). Position the elbow in 45 degrees of flexion to allow for the identification and removal of the 2-0 suture passing needle (500). Insert the 2-0 suture passing needle (500) into the suture passing guide (400) and pass it through until the suture passing needle (500) passes through the hole (209) or stop-hole of the drill guide (200) (FIG. 8) and pierces the skin on the opposite side of the forearm. Ensure the suture passing needle (500) does not hit the ulna upon exit. To avoid this collision, ensure the arm is pronated enough using fluoroscopy to allow the suture passing needle (500) to exit.
10. Pull on the suture passing needle (500) until approximately 4-5 inches of the suture are through the dorsal surface. Proceed to cut the suture proximal to the suture passing needle (500) to maintain as much length as possible. Grasp the suture limbs on both the anterior and dorsal side with hemostats or a needle driver to prevent the suture from sliding back prematurely.
11. Remove the suture passing guide (400) from the suture passing guide tunnel (307) of the locking rod (300). Disengage the locking rod (300) by simultaneously pushing down on the locking rod (300) and the locking rod button (212) on the drill guide (200). Remove the locking rod (300). Rotate the drill guide (200) approximately 90 degrees and remove the drill guide (200) from between the radius and ulna. The 2-0 Suture, looped through the drill guide (200), is thus retrieved. Be sure to remove the suture from the drill guide (200) while maintaining the suture through the tunnel.
12. Use hemostats to grab both ends of the suture loop exiting from the bone tunnels. This will prevent the suture from sliding back prematurely.
13. Remove the hemostat from the sutures. Pass the two suture limbs from the tendon stump through the suture loop that exits through the dorsal aspect of the bicipital tuberosity. This suture limb can be identified as the straight combine leg of the suture. Pull these suture limbs through the bone tunnel from the dorsal to the anterior aspect in preparation for attaching the buttons. Repeat this step if the double-head technique is being completed. Ensure the head of the tendon has been pulled down to the anatomical insertion site.
14. Once the sutures have been passed through the bone tunnel, use the button threader assembly (800) to pass the suture limbs through the holes (811) of the suture button (810) (FIGS. 36 and 37).
15. Once the suture button (810) has been threaded and sits on the periosteum of the tuberosity, position the forearm in approximately 90 degrees of flexion while holding the suture limbs. Apply tension to the suture limbs while rotating the forearm in pronation and supination. This will remove laxity, allowing for proper tensioning.
16. Provisionally lock the suture using a half hitch knot. Confirm that the suture button (810) position is down on the bone surface.
17. Apply tension to the suture limbs with the forearm in approximately 90 degrees of flexion. Use 6-7 half hitch knots to lock the suture.
18. Confirm proper range of motion.
19. Close the wound per surgeon's preference.

Although described above in connection with distal bicep tendon repair and reattachment, these descriptions are not intended to be limiting, as other guide systems can be made in accordance with the description herein, but of different size or scale, for use in other surgical procedures, as desired. As such, although the invention is illustrated and described herein, various modifications and structural changes may be made therein without departing from the spirit of the invention and within the scope and range of equivalents of the claims. Further, any steps described herein may be carried out in any desired order (and any additional steps may be added as desired and/or any steps may be deleted as desired).

Any reference in this specification to “one embodiment,” “an embodiment,” an “example embodiment,” etc., means that a particular feature, structure, or characteristic described in connection with the implementation is included in at least one embodiment of the invention. The appearances of such phrases in various places in the specification are not necessarily referring to the same embodiment. In addition, any elements or limitations of any invention or embodiment thereof disclosed herein can be combined with any and/or all other elements or limitations (individually or in any combination) or any invention or embodiment thereof disclosed herein, and all such combinations are contemplated with the scope of the invention without limitation thereto.

As used herein, the terms “substantial” and “substantially” refer to the complete or nearly complete extent or degrees of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context, which one of ordinary skill in the art would be familiar with. However, generally speaking, the nearness of completion will be so as to have the same overall result as if absolute or total completion were obtained.

The use of term “substantial” or “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

Although described above in connection with particular configurations, these descriptions are not intended to be limiting as various modifications may be made therein without departing from the spirit of the invention and within the scope and range of equivalent of the described embodiments. Encompassed embodiments of the present invention can be used in all surgical applications calling for a guide system.

While a number of embodiments of the present invention have been described, it is understood that these embodiments are illustrative only, and not restrictive, and that many modifications may become apparent to those of ordinary skill in the art. For example, any element described herein may be provided in any desired size (e.g., any element described herein may be provided in any desired custom size or any element described herein may be provided in any desired size selected from a “family” of sizes, such as small, medium, large). Further, one or more of the components may be made from many different suitable materials.

In addition, various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A guide system adapted for drilling through a bone to facilitate a tendon repair and reattachment, the guide system comprising:
a drill guide comprising a body section and a spike section, the body section located proximal of the spike section and comprising a locking rod tunnel, a locking rod ratchet gear, and a guide arm, the locking rod tunnel comprising a proximal end and a distal end, the spike section comprising a hook arm, the hook arm terminating in a hole and a spike adapted to engage a distal side of the bone;
a locking rod adapted to be received by the locking rod tunnel, the locking rod comprising a proximal end, a distal end, and a shaft therebetween, the proximal end of the locking rod comprising a push pad, the distal end of the locking rod comprising a sharp tip adapted to engage a proximal side of the bone, the shaft comprising a plurality of outer ratchet teeth, and a suture passing guide tunnel bored through the shaft;
a suture passing guide adapted to be received by the suture passing guide tunnel, the suture passing guide comprising a proximal end, a distal end, and a shaft therebetween, the shaft comprising a suture passing needle tunnel;
a suture passing needle adapted to be received by the suture passing needle tunnel and to pass through the hole of the hook arm, the suture passing needle comprising a leading end, a trailing end, and a substantially cylindrical body therebetween, the leading end comprising a tip end, the trailing end adapted to receive a suture loop;
the suture loop having an open loop adapted to receive a suture; and
the locking rod ratchet gear adapted to engage the plurality of outer ratchet teeth and to permit the locking rod to slide freely in a direction from the proximal end to the distal end of the locking rod tunnel, and to prevent the locking rod from sliding in a direction from the distal end to the proximal end of the locking rod tunnel.

2. The guide system of claim 1 further comprising a locking rod button on the body section of the drill guide adapted to disengage the locking rod ratchet gear from the plurality of outer ratchet teeth on the shaft of the locking rod and permit the locking rod to slide in the direction from the distal end to the proximal end of the locking rod tunnel.

3. The guide system of claim 1 wherein the suture passing guide tunnel of the locking rod is adapted to receive a drill and to guide the drill in the direction of the hook arm hole.

4. The guide system of claim 1 wherein the hole of the hook arm of the drill guide is a stop-hole.

5. The guide system of claim 4 wherein the suture passing guide tunnel of the locking rod is adapted to receive a drill having a diameter larger than a diameter of the stop-hole, and to guide the drill in the direction of the stop-hole, the stop-hole adapted to limit the travel of the drill through the suture passing guide tunnel.

6. The guide system of claim 4 wherein the shaft of the suture passing guide has a diameter larger than a diameter of the stop-hole and is adapted to be stopped by the stop-hole when the suture passing guide passes through the suture passing guide tunnel.

7. The guide system of claim 1 wherein:
the shaft of the locking rod further comprises a channel at the distal end of the locking rod; and
the body section of the drill guide further comprises a pin adapted to intersect with the locking rod tunnel, the pin further adapted to engage the channel on the shaft of the locking rod to secure and prevent rotation of the locking rod when the locking rod is received by the locking rod tunnel.

8. The guide system of claim 7 wherein the locking rod further comprises:
a distal segment at the distal end of the locking rod, the distal segment comprising a distal end, a proximal end, and a shaft therebetween with a distal segment tunnel bored through the shaft, the distal end of the distal segment comprising the sharp tip of the locking rod, the shaft of the distal segment further comprising the channel adapted to be engaged by the pin of the body section of the drill guide; and
a proximal segment at the proximal end of the locking rod, the proximal segment comprising a distal end, a proximal end, and a shaft therebetween with a proximal segment tunnel bored through the shaft, the proximal end of the proximal segment comprising the push pad of the locking rod, the shaft of the proximal segment further comprising the plurality of outer ratchet teeth of the locking rod;
wherein the proximal end of the distal segment and the distal end of the proximal segment are adapted to removably attach to form the locking rod and align the distal segment tunnel with the proximal segment tunnel to form the suture passing guide tunnel.

9. The guide system of claim 8 wherein the distal end of the proximal segment comprises an internal thread adapted to rotationally engage an external thread of the proximal end of the distal segment.

10. The guide system of claim 8 wherein the channel and the pin are adapted to prevent rotation of the distal segment while allowing linear translation of the distal segment and rotation of the proximal segment.

11. A method of using a guide system to facilitate drilling through a bone and tendon repair and reattachment, the method comprising the steps of:
placing a hook arm of a drill guide around the bone, the drill guide comprising a hook arm terminating in a hole and a body section having a locking rod tunnel, the hole of the hook arm adapted to align with a distal side of the bone;
inserting a locking rod into the locking rod tunnel of the drill guide until the locking rod contacts a proximal side of the bone, the locking rod comprising a suture passing guide tunnel;
inserting a drill into the suture passing guide tunnel of the locking rod and drilling through the bone to form a bone tunnel from the proximal side to the distal side of the bone;
removing the drill from the bone and the suture passing guide tunnel;
inserting a suture passing guide into the suture passing guide tunnel of the locking rod and through the bone tunnel, the suture passing guide comprising a suture passing needle tunnel;
inserting a suture passing needle into the suture passing needle tunnel of the suture passing guide in the bone tunnel, the suture passing needle adapted to receive a suture;
removing the suture passing guide from the bone tunnel and the suture passing guide tunnel of the locking rod while leaving the suture passing needle and the suture in the bone tunnel;
removing the locking rod from the locking rod tunnel of the drill guide; and
removing the drill guide from the bone.

12. The method of claim 11 further comprising:
engaging a sharp tip of the locking rod with the proximal side of the bone when inserting the locking rod into the locking rod tunnel of the drill guide until the locking rod contacts the proximal side of the bone; and
disengaging the sharp tip of the locking rod prior to removing the locking rod from the locking rod tunnel of the drill guide.

13. The method of claim 11 further comprising:
engaging a spike on the hook arm with the distal side of the bone when placing the hook arm of the drill guide around the bone; and
disengaging the spike on the hook arm prior to removing the drill guide from the bone.

14. The method of claim 11 wherein inserting the locking rod into the locking rod tunnel further comprises engaging one of a plurality of outer ratchet teeth on a shaft of the locking rod with a locking rod ratchet gear on the drill guide body section.

15. The method of claim 14 further comprising disengaging the locking rod from the drill guide by pushing on a locking rod button on the drill guide body section to release the locking rod ratchet gear of the drill guide prior to removing the locking rod from the locking rod tunnel.

16. A button threader assembly suitable for threading a suture through a suture button, the button threader assembly comprising:
a suture button comprising two holes each adapted to receive a suture loop;
a button threader handle comprising two substantially pliable loops each adapted to compress through each of the corresponding two holes of the suture button;
a button threader holder comprising a proximal end, a distal end, and a body therebetween, the proximal end comprising two arms adapted to receive the button threader handle and a button slot adapted to receive the suture button, the body comprising two channels adapted to receive the two loops of the button threader handle when each of the two loops is inserted into each of the corresponding two holes of the suture button.

17. The button threader assembly of claim 16 wherein:
each of the two substantially pliable loops of the button threader handle is adapted to receive the suture loop when the button threader handle is received by the two arms of the button threader holder and the two substantially pliable loops are inserted into each of the corresponding two holes of the suture button; and
the button threader handle is adapted to simultaneously pull each of the two substantially pliable loops holding the suture loop through each of the corresponding two holes of the suture button when the button threader handle is detached from the two arms of the button threader holder.
